(19) United States
(12) Patent Application Publication
(10) Pub. No.: US 2026/0355091 A1
(43) Pub. Date: Nov. 12, 2026

(54) Personal Care Compositions

(71) Applicant: Colgate-Palmolive Company, New York, NY (US)

(72) Inventors: Aaron COHEN, New Brunswick, NJ (US); Junhong MAO, Plainsboro, NJ (US); Thomas BOYD, Metuchen, NJ (US)

(73) Assignee: Colgate-Palmolive Company, New York, NY (US)

(21) Appl. No.: 19/126,810

(22) PCT Filed: Dec. 13, 2023
(86) PCT No.: PCT/US2023/083861

§ 371 Date: May 2, 2025

Related U.S. Application Data
(60) Provisional application No. 63/432,286, filed on Dec. 13, 2022.

(51) Int. Cl. A61K 8/67; A61K 8/02; A61K 8/891; A61K 8/92; A61Q 19/00

(52) U.S. Cl. CPC A61K 8/671; A61K 8/0279; A61K 8/891; A61K 8/92; A61Q 19/00; A61K 2800/31; A61K 2800/34

(57) ABSTRACT

Personal care compositions and methods for improving skin are disclosed. A personal care composition according to an aspect of the disclosure may include a delivery system comprising a retinoid, or derivative thereof, incorporated in a delivery vehicle and a cosmetically acceptable carrier that is anhydrous and has a low water content. A method of treating a skin condition according to an aspect of the disclosure may include applying the personal care composition to a skin surface of a subject in need thereof.

### CROSS-REFERENCE TO RELATED APPLICATIONS

This application claims the benefit of priority from U.S. Provisional Application No. 63/432,286, entitled PERSONAL CARE COMPOSITIONS, filed Dec. 13, 2022, the contents of which are hereby incorporated by reference herein in their entireties.

### FIELD OF THE DISCLOSURE

The present disclosure relates to personal care compositions containing a retinoid in an anhydrous carrier or a carrier having a low water content. Aspects of the disclosure also relate to methods for improving skin using such personal care compositions.

### BACKGROUND

Retinol is a skincare active present in many personal care products. It is widely known for its anti-aging and anti-acne benefits via its increase in cellular turnover and overall improvement in skin health. It can be challenging to achieve optimized delivery of retinol into the skin, as one is concerned with not only the total retinol level delivered into the skin, but also the rate at which that amount of retinol is delivered into the skin. Delivering a large amount of retinol too quickly can result in reddened, irritated skin. Therefore, it is desirable to deliver an optimized level of retinol via a sustained rate of release to minimize potential skin irritation.

Along with water, decamethylcyclopentasiloxane (abbreviated as D5) is another common cosmetic ingredient in topical skincare products. D5 is a low molecular weight organosilicon compound whose cosmetically elegant skin feel is enjoyed by consumers.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The present disclosure relates to personal care compositions containing an anhydrous delivery system for active(s) in a carrier having a low water content. The inventors have unexpected discovered that the combination of an anhydrous delivery system and a carrier having a low water content delivers a surprisingly higher level of active(s) into the skin, as compared to the combination of an aqueous delivery system and a carrier having a high water content. Furthermore, the combination of the anhydrous delivery system and the carrier having a low water content achieves a surprisingly sustained (as opposed to burst) increase of retinol in both the epidermis and dermis of the skin over time.

In accordance with some aspects of the invention, provided are personal care compositions and methods for improving skin. In some embodiments, a personal care composition may include a delivery system comprising a retinoid, or derivative thereof, incorporated in a delivery vehicle. The personal care composition may further include a cosmetically acceptable carrier. The personal care composition may include no more than about 35 wt. % of water based on a total weight of the personal care composition.

### BRIEF DESCRIPTION OF THE DRAWINGS

The features, and advantages of the invention will be apparent from the following more detailed description of certain embodiments of the invention and as illustrated in the accompanying drawings in which:

FIG. 1 shows retinol penetration in epidermis and dermis of the skin at 72 hours after three days of daily dosing associated with an example in accordance with aspects of the present invention and a comparative example.

FIG. 2 shows retinol penetration in epidermis and dermis of the skin at 72 hours after three days of daily dosing associated with comparative examples.

FIG. 3 shows retinol penetration in epidermis and dermis of the skin at 72 hours after three days of daily dosing associated with an example in accordance with aspects of the present invention and comparative examples.

FIGS. 4A-4D show the buildup of retinol in epidermis and dermis of the skin associated with an example in accordance with aspects of the present invention and comparative examples.

FIG. 5 shows total retinol contents in epidermis and dermis of the skin associated with an example in accordance with aspects of the present invention and a comparative example.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5 includes specifically 1, 2, 3, 4 and 5, as well as sub ranges such as 2-5, 3-5, 2-3, 2-4, 1-4, etc.

The term “about” when referring to a number means any number within a range of 10% of the number. For example, the phrase “about 2.0 wt. %” refers to a number between and including 1.800 wt. % and 2.200 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the compositions of the instant disclosure can be free or essentially free of all components and elements positively recited throughout the instant disclosure. In some instances, the compositions of the present disclosure may be substantially free of non-incidental amounts of the ingredient(s) or compound(s) described herein. A non-incidental amount of an ingredient or compound is the amount of that ingredient or compound that is added into the composition by itself. For example, a composition may be substantially free of a non-incidental amount of an ingredient or compound, although such ingredient(s) or compound(s) may be present as part of a raw material that is included as a blend of two or more compounds.

For readability purposes, the chemical functional groups are in their adjective form; for each of the adjective, the word “group” is assumed. For example, the adjective “alkyl” without a nouns thereafter, should be read as “an alkyl group”.

Aspects of the present invention relates to personal care compositions containing an anhydrous delivery system for active(s) in a carrier having a low water content. In accordance with some embodiments, the personal care compositions may include: a delivery system comprising a retinoid, or derivative thereof, incorporated in a delivery vehicle; and a cosmetically acceptable carrier. The personal care composition may include no more than about 35 wt. % of water based on a total weight of the personal care composition. The inventors have unexpected discovered that the combination of an anhydrous delivery system and a carrier having a low water content delivers a surprisingly higher level of active(s) into the skin, e.g., into the epidermis and dermis of the skin, as compared to the combination of an aqueous delivery system and a carrier having a high water content (e.g., above 35 wt. %). Further, the combination of the anhydrous delivery system and the carrier having a low water content achieves a surprisingly sustained (as opposed to burst) increase of retinol in both the epidermis and dermis of the skin over time.

The personal care compositions may be in the form of skin care compositions. Non-limiting examples of the form of the personal care compositions according to aspects of the invention may include a lotion, a cream, a serum, a balm, a spray, a sunscreen, a cosmetic, and the like.

Suitable components, as described herein, may be included or excluded from the formulations for the personal care compositions depending on the specific combination of other ingredients, the form of the personal care compositions, and/or the use of the compositions (e.g., a lotion, cream, spray, etc.).

In some embodiments, the delivery system may include a delivery vehicle having one or more actives incorporated or impregnated in the delivery vehicle. In some embodiments, the delivery vehicle may include porous structures. In some embodiments, the delivery vehicle may include porous particles, such as porous spheres. In some embodiments, the porous particle may have an average particle size, e.g., average diameter, ranging from about 10 μm to about 20 μm, e.g., from about 10 μm to about 18 μm, from about 10 μm to about 16 μm, from about 10 μm to about 14 μm, from about 10 μm to about 12 μm, from about 12 μm to about 20 μm, from about 12 μm to about 18 μm, from about 12 μm to about 16 μm, from about 12 μm to about 14 μm, from about 14 μm to about 20 μm, from about 14 μm to about 18 μm, from about 14 μm to about 16 μm, from about 16 μm to about 20 μm, from about 16 μm to about 18 μm, or from about 18 μm to about 20 μm. One non-limiting exemplary delivery vehicle may include Microsponge®.

The delivery vehicle may be formed of a crosspolymer, such as an acrylate crosspolymer. For example, the delivery vehicle (e.g., the plurality of porous structures) may comprise methyl methacrylate/glycol dimethacrylate crosspolymer, cellulose, allyl methacrylates crosspolymer, lauryl methacrylate/glycol dimethacrylate crosspolymer, or a combination of two or more thereof. In some instances, the delivery vehicle is formed of one or more acrylate crosspolymer consisting of methyl methacrylate/glycol dimethacrylate crosspolymer, cellulose, allyl methacrylates crosspolymer, lauryl methacrylate/glycol dimethacrylate crosspolymer, optionally trace materials, and a combination of two or more thereof. In some embodiments, the plurality of porous structures are formed of one or more acrylate crosspolymer consisting of methyl methacrylate/glycol dimethacrylate crosspolymer, cellulose, allyl methacrylates crosspolymer, lauryl methacrylate/glycol dimethacrylate crosspolymer, optionally trace materials, and a combination of two or more thereof.

In some embodiments, the delivery vehicle may include a polymeric matrix. In some embodiments, the polymeric matrix may include a crosspolymer. In some embodiments, the crosspolymer may include an acrylate polymer. Non-limiting examples of suitable crosspolymers may include methyl methacrylate/glycol dimethacrylate crosspolymer, cellulose, allyl methacrylates crosspolymer, lauryl methacrylate/glycol dimethacrylate crosspolymer, and the like.

Preferably, the delivery vehicle is adapted for containing one or more active in the delivery vehicle. In some instances, the delivery vehicle is loaded with the active(s), such that the active is contained within a pore and/or passageway delineated by the delivery vehicle. The personal care composition may be formulated so as to have a delivery vehicle and an active incorporated in the delivery vehicle that is released at a controlled rate. In some embodiments, the personal care composition is formulated to provide a controlled release, e.g., a sustained release, of the active agent.

In some embodiments, the one or more actives that may be incorporated into the delivery vehicle may include a retinoid or derivative thereof. In some embodiments, the retinoid, or derivative thereof, may include one or more of retinol, retinal, tretinoin (retinoic acid), isotretinoin, alitretinoin, etretinate, acitretin, adapalene, bexarotene, tazarotene, trifarotene, or derivatives thereof. Additionally or alternatively, the one or more actives may comprise vitamin A, vitamin C, an antioxidant, and/or a combination thereof. The antioxidant(s) may be selected from propyl gallate, butylated hydroxytoluene (BHT), and a combination thereof. In some cases, the one or more actives consist of retinoid and/or derivative thereof, vitamin A, an antioxidant, and a combination of two or more thereof.

In some embodiments, the retinoid, or derivative thereof, may be present in the delivery system in an amount ranging from about 20 wt. % to about 25 wt. %, based on the weight of the delivery system. For example, in some embodiments, the retinoid, or derivative thereof, may be present in the delivery system in an amount ranging from about 20 wt. % to about 24 wt. %, from about 20 wt. % to about 23 wt. %, from about 20 wt. % to about 22 wt. %, from about 20 wt. % to about 21 wt. %, from about 21 wt. % to about 25 wt. %, from about 21 wt. % to about 24 wt. %, from about 21 wt. % to about 23 wt. %, from about 21 wt. % to about 22 wt. %, from about 22 wt. % to about 25 wt. %, from about 22 wt. % to about 24 wt. %, from about 22 wt. % to about 23 wt. %, from about 23 wt. % to about 25 wt. %, from about 23 wt. % to about 24 wt. %, or from about 24 wt. % to about 25 wt. %, based on the weight of the delivery system.

The retinoid, or derivative thereof, may be present in an amount ranging from about 0.05 wt. % to about 1 wt. %, based on the total weight of the personal care composition. In some embodiments, the retinoid, or derivative thereof, may be present in an amount ranging from about 0.05 wt. % to about 0.9 wt. %, from about 0.05 wt. % to about 0.8 wt. %, from about 0.05 wt. % to about 0.7 wt. %, from about 0.05 wt. % to about 0.6 wt. %, from about 0.05 wt. % to about 0.5 wt. %, from about 0.05 wt. % to about 0.4 wt. %, from about 0.05 wt. % to about 0.3 wt. %, from about 0.05 wt. % to about 0.2 wt. %, from about 0.05 wt. % to about 0.1 wt. %, from about 0.1 wt. % to about 0.9 wt. %, from about 0.1 wt. % to about 0.8 wt. %, from about 0.1 wt. % to about 0.7 wt. %, from about 0.1 wt. % to about 0.6 wt. %, from about 0.1 wt. % to about 0.5 wt. %, from about 0.1 wt. % to about 0.4 wt. %, from about 0.1 wt. % to about 0.3 wt. %, from about 0.1 wt. % to about 0.2 wt. %, from about 0.2 wt. % to about 0.9 wt. %, from about 0.2 wt. % to about 0.8 wt. %, from about 0.2 wt. % to about 0.7 wt. %, from about 0.2 wt. % to about 0.6 wt. %, from about 0.2 wt. % to about 0.5 wt. %, from about 0.2 wt. % to about 0.4 wt. %, from about 0.2 wt. % to about 0.3 wt. %, from about 0.3 wt. % to about 0.9 wt. %, from about 0.3 wt. % to about 0.8 wt. %, from about 0.3 wt. % to about 0.7 wt. %, from about 0.3 wt. % to about 0.6 wt. %, from about 0.3 wt. % to about 0.5 wt. %, from about 0.3 wt. % to about 0.4 wt. %, from about 0.4 wt. % to about 0.9 wt. %, from about 0.4 wt. % to about 0.8 wt. %, from about 0.4 wt. % to about 0.7 wt. %, from about 0.4 wt. % to about 0.6 wt. %, from about 0.4 wt. % to about 0.5 wt. %, from about 0.5 wt. % to about 0.9 wt. %, from about 0.5 wt. % to about 0.8 wt. %, from about 0.5 wt. % to about 0.7 wt. %, from about 0.5 wt. % to about 0.6 wt. %, from about 0.6 wt. % to about 0.9 wt. %, from about 0.6 wt. % to about 0.8 wt. %, from about 0.6 wt. % to about 0.7 wt. %, from about 0.7 wt. % to about 0.9 wt. %, from about 0.7 wt. % to about 0.8 wt. %, or from about 0.8 wt. % to about 0.9 wt. %, based on the total weight of the personal care composition. In some embodiments, the retinoid, or derivative thereof, may be present in an amount of about 0.5 wt. % based on the total weight of the personal care composition.

In some embodiments, the delivery system further may include at least one of a vitamin A, vitamin C, a penetration enhancer, an antioxidant, a surfactant, or a combination thereof, incorporated in the delivery vehicle. In some embodiments, the antioxidant may include at least one of propyl gallate, butylated hydroxytoluene (BHT), or a combination thereof.

In some embodiments, the personal care composition may further include a cosmetically acceptable carrier. In some embodiments, the cosmetically acceptable carrier may have a viscosity ranging from about 6,200 cP to about 60,000 cP. Suitable viscosity, such as a viscosity ranging from about 6,200 cP to about 60,000 cP, may facilitate optimal delivery of actives into the skin. Carriers that have much higher viscosities may hinder active delivery as the active(s) may be trapped in the carrier. In some embodiments, the cosmetically acceptable carrier may have a viscosity ranging from about 6,200 cP to about 60,000 cP, for example, from about 6,200 cP to about 55,000 cP, from about 6,200 cP to about 50,000 cP, from about 6,200 cP to about 45,000 cP, from about 6,200 cP to about 40,000 cP, from about 6,200 cP to about 35,000 cP, from about 6,200 cP to about 30,000 cP, from about 6,200 cP to about 25,000 cP, from about 6,200 cP to about 20,000 cP, from about 6,200 cP to about 15,000 cP, from about 6,200 cP to about 10,000 cP, from about 8,000 cP to about 60,000 cP, from about 8,000 cP to about 55,000 cP, from about 8,000 cP to about 50,000 cP, from about 8,000 cP to about 45,000 cP, from about 8,000 cP to about 40,000 cP, from about 8,000 cP to about 35,000 cP, from about 8,000 cP to about 30,000 cP, from about 8,000 cP to about 25,000 cP, from about 8,000 cP to about 20,000 cP, from about 8,000 cP to about 15,000 cP, from about 8,000 cP to about 10,000 cP, from about 10,000 cP to about 60,000 cP, from about 10,000 cP to about 55,000 cP, from about 10,000 cP to about 50,000 cP, from about 10,000 cP to about 45,000 cP, from about 10,000 cP to about 40,000 cP, from about 10,000 cP to about 35,000 cP, from about 10,000 cP to about 30,000 cP, from about 10,000 cP to about 25,000 cP, from about 10,000 cP to about 20,000 cP, from about 10,000 cP to about 15,000 cP, from about 15,000 cP to about 60,000 cP, from about 15,000 cP to about 55,000 cP, from about 15,000 cP to about 50,000 cP, from about 15,000 cP to about 45,000 cP, from about 15,000 cP to about 40,000 cP, from about 15,000 cP to about 35,000 cP, from about 15,000 cP to about 30,000 cP, from about 15,000 cP to about 25,000 cP, from about 15,000 cP to about 20,000 cP, from about 20,000 cP to about 60,000 cP, from about 20,000 cP to about 55,000 cP, from about 20,000 cP to about 50,000 cP, from about 20,000 cP to about 45,000 cP, from about 20,000 cP to about 40,000 cP, from about 20,000 cP to about 35,000 cP, from about 20,000 cP to about 30,000 cP, from about 20,000 cP to about 25,000 cP, from about 25,000 cP to about 60,000 cP, from about 25,000 cP to about 55,000 cP, from about 25,000 cP to about 50,000 cP, from about 25,000 cP to about 45,000 cP, from about 25,000 cP to about 40,000 cP, from about 25,000 cP to about 35,000 cP, from about 25,000 cP to about 30,000 cP, from about 30,000 cP to about 60,000 cP, from about 30,000 cP to about 55,000 cP, from about 30,000 cP to about 50,000 cP, from about 30,000 cP to about 45,000 cP, from about 30,000 cP to about 40,000 cP, from about 30,000 cP to about 35,000 cP, from about 35,000 cP to about 60,000 cP, from about 35,000 cP to about 55,000 cP, from about 35,000 cP to about 50,000 cP, from about 35,000 cP to about 45,000 cP, from about 35,000 cP to about 40,000 cP, from about 40,000 cP to about 60,000 cP, from about 40,000 cP to about 55,000 cP, from about 40,000 cP to about 50,000 cP, from about 40,000 cP to about 45,000 cP, from about 45,000 cP to about 60,000 cP, from about 45,000 cP to about 55,000 cP, from about 45,000 cP to about 50,000 cP, from about 50,000 cP to about 60,000 cP, from about 50,000 cP to about 55,000 cP, or from about 55,000 cP to about 60,000 cP. Additionally and/or alternatively, the personal care composition may be formulated such that the cosmetically acceptable carrier, as whole (e.g., when the cosmetically acceptable carrier comprises a plurality of cosmetically acceptable carriers), has a viscosity according to one of the foregoing viscosity ranges disclosed above with respect to cosmetically acceptable carrier. The viscosity of the acceptable carrier may be measured using a Brookfield viscometer. In some embodiments, the viscosity of the acceptable carrier may be measured using a Brookfield viscometer and a spindle number LV1 at an RPM of 20. In other embodiments, the viscosity of the acceptable carrier may be measured using a Brookfield viscometer and a spindle number LV2 at an RPM of 20.

The cosmetically acceptable carrier may be selected from silicones, polyols, monoalcohols, water, and a combination of two or more thereof. Examples of monoalcohols that may be included or excluded from the personal care composition include ethanol, propanol, butanol, or a combination thereof.

In some embodiments, the cosmetically acceptable carrier may include a silicone. For example, the personal care composition may include a cosmetically acceptable carrier comprising a silicone having a viscosity according to one of the foregoing viscosity ranges disclosed above with respect to the cosmetically acceptable carrier. The silicone may be present in an amount ranging from about 55 wt. % to about 95 wt. %, based on the total weight of the personal care composition. For example, in some embodiments, the silicone may be present in an amount ranging from about 55 wt. % to about 90 wt. %, from about 55 wt. % to about 85 wt. %, from about 55 wt. % to about 80 wt. %, from about 55 wt. % to about 75 wt. %, from about 55 wt. % to about 70 wt. %, from about 55 wt. % to about 65 wt. %, from about 55 wt. % to about 60 wt. %, from about 60 wt. % to about 95 wt. %, from about 60 wt. % to about 90 wt. %, from about 60 wt. % to about 85 wt. %, from about 60 wt. % to about 80 wt. %, from about 60 wt. % to about 75 wt. %, from about 60 wt. % to about 70 wt. %, from about 60 wt. % to about 65 wt. %, from about 65 wt. % to about 95 wt. %, from about 65 wt. % to about 90 wt. %, from about 65 wt. % to about 85 wt. %, from about 65 wt. % to about 80 wt. %, from about 65 wt. % to about 75 wt. %, from about 65 wt. % to about 70 wt. %, from about 70 wt. % to about 95 wt. %, from about 70 wt. % to about 90 wt. %, from about 70 wt. % to about 85 wt. %, from about 70 wt. % to about 80 wt. %, from about 70 wt. % to about 75 wt. %, from about 75 wt. % to about 95 wt. %, from about 75 wt. % to about 90 wt. %, from about 75 wt. % to about 85 wt. %, from about 75 wt. % to about 80 wt. %, from about 80 wt. % to about 95 wt. %, from about 80 wt. % to about 90 wt. %, from about 80 wt. % to about 85 wt. %, from about 85 wt. % to about 95 wt. %, from about 85 wt. % to about 90 wt. %, or from about 90 wt. % to about 95 wt. %, based on the total weight of the personal care composition.

In some embodiments, the silicone may include a silicone selected from dimethicone, cetearyl methicone, bis-stearyl dimethicone, cyclomethicone, polysilicone-11, phenyl trimethicone, trimethylsilylamodimethicone, phenyl dimethicones, phenyl(trimethylsiloxy)diphenylsiloxanes, diphenyl dimethicones, diphenyl(methyldiphenyl)trisiloxanes, stearoxytrimethylsilane, and a combination of two or more thereof. In some embodiments, the skin conditioning agent comprises dimethicone, cetearyl methicone, bis-stearyl dimethicone, or a combination of two or more thereof.

In some embodiments, the silicone may include a “silicone oil”. The term “silicone oil” relates to oil comprising at least one silicon atom, and especially at least one Si—O group. Non-limiting examples of silicone oils include dimethicone, cyclomethicone, polysilicone-11, phenyl trimethicone, trimethylsilylamodimethicone, and stearoxytrimethylsilane. In some cases, the personal care composition includes dimethicone, and optionally additional oils, including additional silicone oils. Typically, the one or more silicone oils is a non-volatile silicone oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

In some embodiments, the cosmetically acceptable carrier may include at least one of cyclopentasiloxane, dimethicone, dimethicone crosspolymer, or dimethicone/vinyl dimethicone crosspolymer.

The cosmetically acceptable carrier may comprise one or more humectant in an amount from about 1 to about 25 wt. %, based on the total weight of the personal care composition. In some instances, the total amount of humectant(s) present in the personal care composition is about 1 to about 25 wt. %, about 2 to about 25 wt. %, about 3 to about 25 wt. %, about 4 to about 25 wt. %, about 5 to about 25 wt. %, about 7.5 to about 25 wt. %, about 10 to about 25 wt. %, about 15 to about 25 wt. %, about 20 to about 25 wt. %; about 1 to about 20 wt. %, about 2 to about 20 wt. %, about 3 to about 20 wt. %, about 4 to about 20 wt. %, about 5 to about 20 wt. %, about 7.5 to about 20 wt. %, about 10 to about 20 wt. %, about 15 to about 20 wt. %; about 1 to about 15 wt. %, about 2 to about 15 wt. %, about 3 to about 15 wt. %, about 4 to about 15 wt. %, about 5 to about 15 wt. %, about 7.5 to about 15 wt. %, about 10 to about 15 wt. %; about 1 to about 10 wt. %, about 2 to about 10 wt. %, about 3 to about 10 wt. %, about 4 to about 10 wt. %, about 5 to about 10 wt. %, about 7.5 to about 10 wt. %; about 1 to about 7.5 wt. %, about 2 to about 7.5 wt. %, about 3 to about 7.5 wt. %, about 4 to about 7.5 wt. %, about 5 to about 7.5 wt. %; about 1 to about 5 wt. %, about 2 to about 5 wt. %, about 3 to about 5 wt. %, about 4 to about 5 wt. %; about 1 to about 4 wt. %, about 2 to about 4 wt. %, about 3 to about 4 wt. %, including all ranges and subranges therebetween, based on the total weight of the personal care composition.

Examples of humectants that may be incorporated into the personal care composition include, but are not limited to, polyhydric alcohols such as glycerin, butylene glycol, 1,3-propanediol, sorbitol, xylitol or low molecular weight polyethylene glycols (PEGs), polyoxyethylenes or combinations thereof. In some embodiments, the humectant agents are sorbitol (preferably, non-crystal sorbitol), butylene glycol, 1,3-propanediol, glycerin (e.g., vegetable refined glycerin), polyoxyethylene glycol or combinations thereof. In at least one embodiment, the humectants are chosen from glycerin, butylene glycol and 1,3-propanediol. Typically, the humectant is vegetable refined glycerin. Other suitable humectants include, but are not limited to, polyglutamic acid, glycerol and its derivatives, urea and its derivatives, sodium pidolate, xylitol, serine, sodium lactate, ectoin and its derivatives, chitosan and its derivatives, collagen, plankton, an extract of Imperata cylindra, beta-glucan, and/or rose hip oil.

In certain embodiments, the personal care composition includes a humectant selected from sorbitol, butylene glyco1,1,3-propanediol, glycerin, polyoxyethylene glycol, polyglutamic acid, or a combination of two or more thereof. In at least one preferred embodiment, the personal care composition includes a cosmetically acceptable carrier comprising a humectant having a viscosity according to one of the foregoing viscosity ranges disclosed above with respect to the cosmetically acceptable carrier.

In some embodiments, the personal care composition may be substantially free of select silicones. In some embodiments, the personal care composition may be substantially free of cyclic silicones. For instance, the personal care composition may contain cyclic silicones in an amount of about 3 wt. % or less, about 2 wt. % or less, about 1 wt. % or less, about 0.5 wt. % or less, about 0.1 wt. % or less, about 0.05 wt. % or less or about 0.01 wt. % or less of cyclic silicones. In at least one embodiment, the personal care composition has about 0 wt. % or 0 wt. % of cyclic silicones. In some embodiments, the personal care composition may be substantially free of decamethylcyclopentasiloxane. For instance, the personal care composition may contain decamethylcyclopentasiloxane in an amount of about 3 wt. % or less, about 2 wt. % or less, about 1 wt. % or less, about 0.5 wt. % or less, about 0.1 wt. % or less, about 0.05 wt. % or less or about 0.01 wt. % or less of decamethylcyclopentasiloxane. In at least one embodiment, the personal care composition has about 0 wt. % or 0 wt. % of decamethylcyclopentasiloxane.

In some embodiments, the personal care composition may be substantially free of parabens, preferably free of parabens.

In some embodiments, the cosmetically acceptable carrier may include a nonionic surfactant. In some embodiments, the nonionic surfactant may include at least one of laureth-4, laureth-23, C12-14 alketh-12 (or C12-14 pareth-12), polysorbates (e.g., Tween® surfactants), polyethoxy fatty ethers (e.g., Brij® surfactants), sorbitan fatty acid esters (e.g., Span® surfactants), or a combination thereof.

As discussed above, in some embodiments, the personal care composition may be substantially anhydrous, preferably anhydrous. The term “anhydrous” used herein means that no water is added to the composition and the total water content of the composition is less than about 5 wt. %, preferably less than about 3 wt. %, about 2 wt. %, about 1 wt. %, about 0.5 wt. %, e.g., less than 0.1 wt. %, relative to the total weight of the personal care composition, and in some embodiments, the personal care composition may contain no water. In some embodiments, at least one of the delivery system or the cosmetically acceptable carrier may be substantially anhydrous, preferably anhydrous. In some embodiments, the delivery system may be substantially anhydrous, preferably anhydrous. In some embodiments, the cosmetically acceptable carrier may be substantially anhydrous, preferably anhydrous.

In some embodiments, the personal care composition may include a low water content. In some embodiments, the personal care composition may include no more than about 35 wt. % of water based on a total weight of the personal care composition. For example, in some embodiments, water may be present in the personal care composition in an amount ranging from about 1 wt. % to about 35 wt. %, from about 1 wt. % to about 30 wt. %, from about 1 wt. % to about 25 wt. %, from about 1 wt. % to about 20 wt. %, from about 1 wt. % to about 15 wt. %, from about 1 wt. % to about 10 wt. %, from about 1 wt. % to about 5 wt. %, from about 5 wt. % to about 35 wt. %, from about 5 wt. % to about 30 wt. %, from about 5 wt. % to about 25 wt. %, from about 5 wt. % to about 20 wt. %, from about 5 wt. % to about 15 wt. %, from about 5 wt. % to about 10 wt. %, from about 10 wt. % to about 35 wt. %, from about 10 wt. % to about 30 wt. %, from about 10 wt. % to about 25 wt. %, from about 10 wt. % to about 20 wt. %, from about 10 wt. % to about 15 wt. %, from about 15 wt. % to about 35 wt. %, from about 15 wt. % to about 30 wt. %, from about 15 wt. % to about 25 wt. %, from about 15 wt. % to about 20 wt. %, from about 20 wt. % to about 35 wt. %, from about 20 wt. % to about 30 wt. %, from about 20 wt. % to about 25 wt. %, from about 25 wt. % to about 35 wt. %, from about 25 wt. % to about 30 wt. %, or from about 30 wt. % to about 35 wt. % based on the total weight of the personal care composition.

In some embodiments, the cosmetically acceptable carrier may include a low water content. In some embodiments, the cosmetically acceptable carrier may include no more than about 35 wt. % of water based on a weight of the cosmetically acceptable carrier. For example, in some embodiments, water may be present in the cosmetically acceptable carrier in an amount ranging from about 1 wt. % to about 35 wt. %, from about 1 wt. % to about 30 wt. %, from about 1 wt. % to about 25 wt. %, from about 1 wt. % to about 20 wt. %, from about 1 wt. % to about 15 wt. %, from about 1 wt. % to about 10 wt. %, from about 1 wt. % to about 5 wt. %, from about 5 wt. % to about 35 wt. %, from about 5 wt. % to about 30 wt. %, from about 5 wt. % to about 25 wt. %, from about 5 wt. % to about 20 wt. %, from about 5 wt. % to about 15 wt. %, from about 5 wt. % to about 10 wt. %, from about 10 wt. % to about 35 wt. %, from about 10 wt. % to about 30 wt. %, from about 10 wt. % to about 25 wt. %, from about 10 wt. % to about 20 wt. %, from about 10 wt. % to about 15 wt. %, from about 15 wt. % to about 35 wt. %, from about 15 wt. % to about 30 wt. %, from about 15 wt. % to about 25 wt. %, from about 15 wt. % to about 20 wt. %, from about 20 wt. % to about 35 wt. %, from about 20 wt. % to about 30 wt. %, from about 20 wt. % to about 25 wt. %, from about 25 wt. % to about 35 wt. %, from about 25 wt. % to about 30 wt. %, or from about 30 wt. % to about 35 wt. % based on the weight of the cosmetically acceptable carrier.

In some embodiments, the cosmetically acceptable carrier may include a structuring agent. As discussed above, the delivery vehicle may include a polymeric matrix that may include a crosspolymer. The structuring agent may allow the crosslinked delivery vehicle to be dispersed throughout the cosmetically acceptable carrier without settling over extended period of time.

In some embodiments, the structuring agent may be present in an amount ranging from about 5 wt. % to about 65 wt. % based on the total weight of the personal care composition. For example, in some embodiments, the structuring agent may be present in an amount ranging from about 5 wt. % to about 60 wt. %, from about 5 wt. % to about 55 wt. %, from about 5 wt. % to about 50 wt. %, from about 5 wt. % to about 45 wt. %, from about 5 wt. % to about 40 wt. %, from about 5 wt. % to about 35 wt. %, from about 5 wt. % to 30 wt. %, from about 5 wt. % to about 25 wt. %, from about 5 wt. % to about 20 wt. %, from about 5 wt. % to about 15 wt. %, from about 5 wt. % to about 10 wt. %, from about 10 wt. % to about 65 wt. %, from about 10 wt. % to about 60 wt. %, from about 10 wt. % to about 55 wt. %, from about 10 wt. % to about 50 wt. %, from about 10 wt. % to about 45 wt. %, from about 10 wt. % to about 40 wt. %, from about 10 wt. % to about 35 wt. %, from about 10 wt. % to 30 wt. %, from about 10 wt. % to about 25 wt. %, from about 10 wt. % to about 20 wt. %, from about 10 wt. % to about 15 wt. %, from about 15 wt. % to about 65 wt. %, from about 15 wt. % to about 60 wt. %, from about 15 wt. % to about 55 wt. %, from about 15 wt. % to about 50 wt. %, from about 15 wt. % to about 45 wt. %, from about 15 wt. % to about 40 wt. %, from about 15 wt. % to about 35 wt. %, from about 15 wt. % to 30 wt. %, from about 15 wt. % to about 25 wt. %, from about 15 wt. % to about 20 wt. %, from about 20 wt. % to about 65 wt. %, from about 20 wt. % to about 60 wt. %, from about 20 wt. % to about 55 wt. %, from about 20 wt. % to about 50 wt. %, from about 20 wt. % to about 45 wt. %, from about 20 wt. % to about 40 wt. %, from about 20 wt. % to about 35 wt. %, from about 20 wt. % to 30 wt. %, from about 20 wt. % to about 25 wt. %, from about 25 wt. % to about 65 wt. %, from about 25 wt. % to about 60 wt. %, from about 25 wt. % to about 55 wt. %, from about 25 wt. % to about 50 wt. %, from about 25 wt. % to about 45 wt. %, from about 25 wt. % to about 40 wt. %, from about 25 wt. % to about 35 wt. %, from about 25 wt. % to 30 wt. %, from about 30 wt. % to about 65 wt. %, from about 30 wt. % to about 60 wt. %, from about 30 wt. % to about 55 wt. %, from about 30 wt. % to about 50 wt. %, from about 30 wt. % to about 45 wt. %, from about 30 wt. % to about 40 wt. %, from about 30 wt. % to about 35 wt. %, from about 35 wt. % to about 65 wt. %, from about 35 wt. % to about 60 wt. %, from about 35 wt. % to about 55 wt. %, from about 35 wt. % to about 50 wt. %, from about 35 wt. % to about 45 wt. %, from about 35 wt. % to about 40 wt. %, from about 40 wt. % to about 65 wt. %, from about 40 wt. % to about 60 wt. %, from about 40 wt. % to about 55 wt. %, from about 40 wt. % to about 50 wt. %, from about 40 wt. % to about 45 wt. %, from about 45 wt. % to about 65 wt. %, from about 45 wt. % to about 60 wt. %, from about 45 wt. % to about 55 wt. %, from about 45 wt. % to about 50 wt. %, from about 50 wt. % to about 65 wt. %, from about 50 wt. % to about 60 wt. %, from about 50 wt. % to about 55 wt. %, from about 55 wt. % to about 65 wt. %, from about 55 wt. % to about 60 wt. %, or from about 60 wt. % to about 65 wt. % based on the total weight of the personal care composition.

In some embodiments, the structuring agent may include a polymer. In some embodiments, the structuring agent may include at least one of a wax or a crosspolymer. In some embodiments, the structuring agent may include dimethicone crosspolymer, dimethicone/vinyl dimethicone crosspolymer, polysilicone-11, or a combination of two or more thereof. Examples of wax structuring agents include silicone wax, paraffin wax, microcrystalline wax other than paraffin, and polyethylene wax. Silicone wax structuring agents include, e.g., cetyl-, cetearyl-, stearyl-, behenyl-, C16-18-, C20-24-, C24-C28-, and C30-C45 methyl dimethicones such as, for example, for example, stearyl dimethicone, available from Dow Corning as DC-2503 wax, C16-18 alkyl dimethicone available from Momentive Performance Materials as SF-1632, CF40-45 alkyl dimethicone available from Momentive Performance Materials as SF-1642, alkoxysilanes, such as, for example stearoxytrimethyl silane, available from Dow Corning as DC Q5-0158A wax, behenoxydimethicone available from Evonik as Abil® Wax 2440; polyether silanes such as, for example, bis PEG-18-methyl ether dimethyl silane such as, for example, Dow Corning® 2501, and the like, and mixtures thereof.

Paraffin wax is generally a hydrocarbon wax, typically derived from petroleum or petrolatum. In one form, paraffin wax is predominantly a mixture of aliphatic alkanes that are themselves predominantly linear or normal alkanes, commonly as a mixture of C20 to C40 alkanes, which form is commonly referred to as “hydrocarbon paraffin’ or “linear paraffin”, notwithstanding that a minor portion of the paraffin wax may be branched. Forms in which branched alkanes predominate are often referred to as isoparaffin. The melting point of the paraffin wax used herein typically is at least 55 °C, and normally less than 70 °C. Paraffin wax having a melting point in a temperature range of from 55 °C to 65 °C is of interest in one or more embodiments, with paraffin wax having a melting point in a temperature range of from 60 °C to 65 °C being of particular interest.

Microcrystalline waxes are commonly produced by the de-oiling of petrolatum. Compared to paraffin wax, microcrystalline wax commonly has a greater proportion of branched alkanes and a larger portion of higher molecular weight alkanes; compared to paraffin wax, microcrystalline is generally a less brittle, more malleable wax. When employed the polyethylene wax used herein typically has a nominal molecular weight of from 200 to 2000 Daltons more particularly, from 200 to 1000 Daltons, and even more particularly from 300 to 600 Daltons.

Silicone elastomers for use herein include cross-linked polydimethyl or polymonomethyl siloxanes optionally having end groups such as hydroxyl or methyl. Preferred silicone elastomers for use in the invention are cross-linked polydiorganosiloxanes, preferably derived from suitable combinations of R₃SiO_{0.5} units and R₂SiO units where each R independently represents an alkyl, alkenyl (e.g. vinyl), alkaryl, aralkyl, or aryl (e.g. phenyl) group. R is most preferably methyl. Such elastomers are described, for example, in U.S. Pat. No. 5,654,362.

As noted above, the personal care composition may comprise an acceptable carrier that is a structuring agent having a viscosity ranging from about 6,200 cP to about 60,000 cP, for example, from about 6,200 cP to about 55,000 cP, from about 6,200 cP to about 50,000 cP, from about 6,200 cP to about 45,000 cP, from about 6,200 cP to about 40,000 cP, from about 6,200 cP to about 35,000 cP, from about 6,200 cP to about 30,000 cP, from about 6,200 cP to about 25,000 cP, from about 6,200 cP to about 20,000 cP, from about 6,200 cP to about 15,000 cP, from about 6,200 cP to about 10,000 cP, from about 8,000 cP to about 60,000 cP, from about 8,000 cP to about 55,000 cP, from about 8,000 cP to about 50,000 cP, from about 8,000 cP to about 45,000 cP, from about 8,000 cP to about 40,000 cP, from about 8,000 cP to about 35,000 cP, from about 8,000 cP to about 30,000 cP, from about 8,000 cP to about 25,000 cP, from about 8,000 cP to about 20,000 cP, from about 8,000 cP to about 15,000 cP, from about 8,000 cP to about 10,000 cP, from about 10,000 cP to about 60,000 cP, from about 10,000 cP to about 55,000 cP, from about 10,000 cP to about 50,000 cP, from about 10,000 cP to about 45,000 cP, from about 10,000 cP to about 40,000 cP, from about 10,000 cP to about 35,000 cP, from about 10,000 cP to about 30,000 cP, from about 10,000 cP to about 25,000 cP, from about 10,000 cP to about 20,000 cP, from about 10,000 cP to about 15,000 cP, from about 15,000 cP to about 60,000 cP, from about 15,000 cP to about 55,000 cP, from about 15,000 cP to about 50,000 cP, from about 15,000 cP to about 45,000 cP, from about 15,000 cP to about 40,000 cP, from about 15,000 cP to about 35,000 cP, from about 15,000 cP to about 30,000 cP, from about 15,000 cP to about 25,000 cP, from about 15,000 cP to about 20,000 cP, from about 20,000 cP to about 60,000 cP, from about 20,000 cP to about 55,000 cP, from about 20,000 cP to about 50,000 cP, from about 20,000 cP to about 45,000 cP, from about 20,000 cP to about 40,000 cP, from about 20,000 cP to about 35,000 cP, from about 20,000 cP to about 30,000 cP, from about 20,000 cP to about 25,000 cP, from about 25,000 cP to about 60,000 cP, from about 25,000 cP to about 55,000 cP, from about 25,000 cP to about 50,000 cP, from about 25,000 cP to about 45,000 cP, from about 25,000 cP to about 40,000 cP, from about 25,000 cP to about 35,000 cP, from about 25,000 cP to about 30,000 cP, from about 30,000 cP to about 60,000 cP, from about 30,000 cP to about 55,000 cP, from about 30,000 cP to about 50,000 cP, from about 30,000 cP to about 45,000 cP, from about 30,000 cP to about 40,000 cP, from about 30,000 cP to about 35,000 cP, from about 35,000 cP to about 60,000 cP, from about 35,000 cP to about 55,000 cP, from about 35,000 cP to about 50,000 cP, from about 35,000 cP to about 45,000 cP, from about 35,000 cP to about 40,000 cP, from about 40,000 cP to about 60,000 cP, from about 40,000 cP to about 55,000 cP, from about 40,000 cP to about 50,000 cP, from about 40,000 cP to about 45,000 cP, from about 45,000 cP to about 60,000 cP, from about 45,000 cP to about 55,000 cP, from about 45,000 cP to about 50,000 cP, from about 50,000 cP to about 60,000 cP, from about 50,000 cP to about 55,000 cP, or from about 55,000 cP to about 60,000 cP. The viscosity of the acceptable carrier may be measured using a Brookfield viscometer. In some embodiments, the viscosity of the acceptable carrier may be measured using a Brookfield viscometer and a spindle number LV1 at an RPM of 20. In other embodiments, the viscosity of the acceptable carrier may be measured using a Brookfield viscometer and a spindle number LV2 at an RPM of 20.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

In certain embodiments, the personal care composition has a pH of from about 3.5 to about 10.0. In other embodiments, the personal care composition has a pH of from about 3.5 to about 8.0. In some embodiments, the personal care composition has a pH of from about 3.5 to about 7.5. In other embodiments, the personal care composition has a pH of from about 3.5 to about 7.0. In further embodiments, the personal care composition has a pH of from about 3.5 to about 6.5. Still other embodiments provide personal care compositions having a pH of from about 3.5 to about 6.0. While other embodiments provide personal care compositions having a pH of from about 3.5 to about 5.5. Yet other embodiments provide personal care compositions having a pH of from about 3.5 to about 5.0. In certain embodiments, the personal care composition has a pH of from about 3.75 to about 4.5.

Aspects of the present invention further relates to method of treating a skin condition. The method may include applying the personal care composition described herein to a skin surface of a subject in need thereof. The skin condition that may be treated may include aging, photoaged and/or photodamaged skin, acne, skin darkening/pigmentation, uneven skin tone, skin pores, deteriorated skin firmness, deteriorated texture, deteriorated skin elasticity, skin redness, and/or deteriorated cell turnover.

Implementation of the present disclosure is provided by way of the following examples. The examples serve to illustrate the technology without being limiting in nature.

### EXAMPLES

A first non-limiting exemplary personal care composition (Example 1) and a first, second, third, and fourth comparative personal care compositions (Comparative Example A, Comparative Example B, Comparative Example C, and Comparative Example D) were prepared as follows.

### Example 1

A first non-limiting exemplary personal care composition (Example 1) was prepared in accordance with aspects of the invention. The first exemplary personal care composition included a delivery system having retinol loaded in a delivery vehicle having a porous structure, specifically, Microsponge®, and a carrier with a low water content. The loading amount of the retinol was 20 wt. % to 25 wt. % based on a total weight of the delivery system, and the delivery system included substantially no water. The carrier included no more than 15 wt. % of water (about 10 wt. % to 15 wt. % of water) based on a total weight of the carrier. The carrier further included a dimethicone crosspolymer as a structuring agent and had a viscosity of about 30,000 cP. The amounts of the retinol-containing delivery system and the carrier were selected such that the resultant first exemplary personal care composition included 0.5 wt. % of retinol and no more than 15 wt. % of water based on a total weight of the first exemplary personal care composition.

### Comparative Example A

A first comparative personal care composition (Comparative Example A) was prepared as follows. The first comparative personal care composition included a delivery system having retinol loaded in vesicles formed via self-assembly of amphiphilic surfactants in water. The first comparative personal care composition further included an oil/water emulsion as the carrier incorporating the delivery system. Thus, both the delivery system and the carrier included water in the first comparative personal care composition. The carrier had a viscosity of about 16,550 to about 18,500 cP. The first comparative personal care composition included 0.5 wt. % of retinol and 57.46 wt. % of water based on a total weight of the first comparative personal care composition.

### Comparative Example B

A second comparative personal care composition (Comparative Example B) was prepared as follows. The second comparative personal care composition included a delivery system having retinol loaded in a delivery vehicle having a porous structure, specifically, Microsponge®. The loading amount of the retinol was 20 wt. % to 25 wt. % based on a total weight of the delivery system, and the delivery system included substantially no water. The second comparative personal care composition further included an oil/water emulsion as the carrier. The carrier had a viscosity of about 16,550 to about 18,500 cP. The second comparative personal care composition included about 0.5 wt. % of retinol and about 50 wt. % water, based on a total weight of the second comparative personal care composition.

### Comparative Example C

A third comparative personal care composition (Comparative Example C) was prepared as follows. The third comparative personal care composition included a delivery system having retinol loaded in a delivery vehicle having a porous structure, specifically, Microsponge®. The loading amount of the retinol was 20 wt. % to 25 wt. % based on a total weight of the delivery system, and the delivery system included substantially no water. The third comparative personal care composition further included an anhydrous carrier incorporating the delivery system. The carrier further included polysilicone-11 as a structuring agent and had a viscosity of from about 85,000 cP to about 129,000 cP. The third comparative personal care composition included 0.5 wt. % of retinol based on a total weight of the third comparative personal care composition and contained no water.

### Comparative Example D

A fourth comparative personal care composition (Comparative Example D) was prepared as follows. The fourth comparative personal care composition included a delivery system having retinol loaded in a delivery vehicle having a porous structure, specifically, Microsponge®. The loading amount of the retinol was 20 wt. % to 25 wt. % based on a total weight of the delivery system, and the delivery system included substantially no water. The fourth comparative personal care composition further included an anhydrous carrier incorporating the delivery system. The carrier further included polysilicone-11 as a structuring agent and had a viscosity of from about 74,000 cP to about 97,000 cP. The fourth comparative personal care composition included 0.5 wt. % of retinol based on a total weight of the fourth comparative personal care composition and contained no water.

### Experiment 1—Total Retinol Penetration

In vitro penetration tests (IVPT) of retinol delivery were conducted on human skin explants. Specifically, samples of Example 1 and Comparative Example A were applied to NativeSkin® human skin explants (diameter of 1.5 cm) from Genoskin Inc. at 0, 24, and 48 hours, and then the retinol contents in the epidermis and dermis were determined at 72 hours via high-performance liquid chromatography (HPLC) quantitative analysis for each sample.

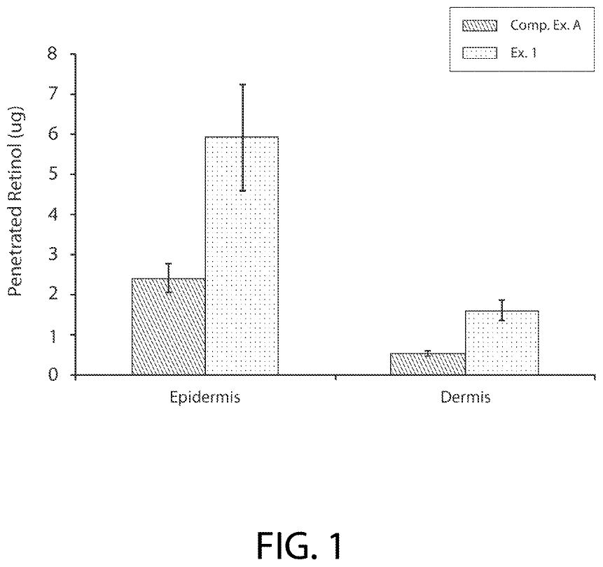

FIG. 1 shows retinol penetration, measured in μg of delivered retinol, in both the epidermis and dermis at 72 hours after three days of daily dosing. It has been surprisingly discovered that Example 1 significantly outperformed Comparative Example A in both the epidermis and dermis. This indicates that the combination of an anhydrous delivery system having retinol-loaded a delivery vehicle having a porous structure, such as Microsponge®, and a low-water-content carrier could deliver a significantly higher level of retinol, when compared to the combination of an aqueous delivery system having retinol-loaded vesicles that self-assembly in water and an oil/water emulsion carrier.

### Experiment 2—Total Retinol Penetration

Samples of Comparative Example A, Comparative Example B, and Comparative Example C were applied to NativeSkin® human skin explants (diameter of 1.1 cm) from Genoskin, Inc. at 0, 24, and 48 hours, and then the retinol contents in the epidermis and dermis were determined at 72 hours via HPLC quantitative analysis for each sample.

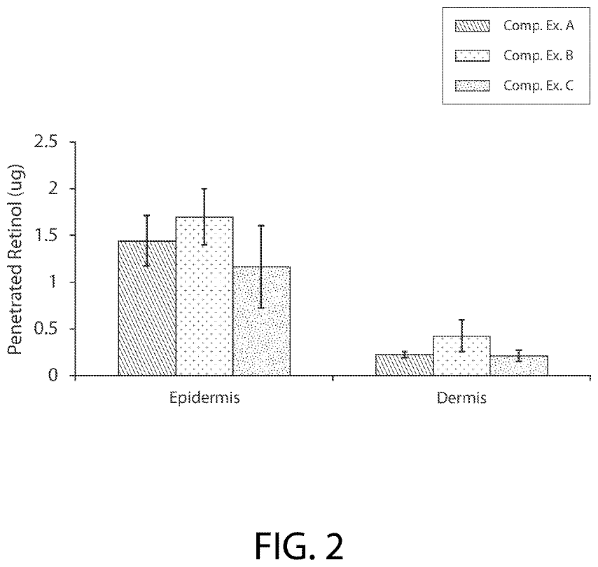

FIG. 2 shows retinol penetration, measured in μg of delivered retinol, in both the epidermis and dermis. It is noted that in Experiment 1, medium sized skin explants (diameter of 1.5 cm) were used, while in Experiment 2, small sized skin explants (diameter of 1.1 cm) were used, likely resulting in the lower amount of retinol penetration observed for Comparative Example A in Experiment 2 than in Experiment 1.

Based on the results from Experiment 1 and Experiment 2, Table 1 below summarizes the normalized total amount of retinol penetration and change (%) in retinol penetration in the epidermis, both of which are calculated using the total amount of retinol penetration associated with Comparative Example A as the reference. Table 2 below summarizes the normalized total amount of retinol penetration and change (%) in retinol penetration in the dermis, both of which are calculated using the total amount of retinol penetration of Comparative Example A as the reference.

TABLE 1
Retinol Penetration in Epidermis

| | | Comp. | | Comp. | Comp. |
|---|---|---|---|---|---|
| | | Ex. A | Ex. 1 | Ex. B | Ex. C |
| Experiment 1 | Total | 1 | 2.44 | — | — |
| | Change | 0% | 144% | — | — |
| Experiment 2 | Total | 1 | — | 1.18 | 0.81 |
| | Change | 0% | — | 18% | −19% |

TABLE 2
Retinol Penetration in Dermis

| | | Comp. | | Comp. | Comp. |
|---|---|---|---|---|---|
| | | Ex. A | Ex. 1 | Ex. B | Ex. C |
| Experiment 1 | Total | 1 | 3 | — | — |
| | Change | 0% | 200% | — | — |
| Experiment 2 | Total | 1 | — | 1.87 | 0.95 |
| | Change | 0% | — | 87% | −5% |

Comparing results from Experiment 2 to the results from Experiment 1, the changes in retinol penetration associated with Comparative Example B and Comparative Example C in Experiment 2 were less dramatic than the change in retinol penetration associated with Example 1 in Experiment 1. Specifically, Comparative Example B showed a consistent, but small increase in overall retinol delivery in both the epidermis and dermis (18% increase and 87% increase in the epidermis and dermis, respectively). In contrast, Example 1 showed not only a consistent, but also a more significant increase in overall retinol delivery in both the epidermis and dermis (144% increase and 200% increase in the epidermis and dermis, respectively). Thus, although using a delivery system containing retinol-loaded a delivery vehicle having a porous structure, such as Microsponge®, may increase retinol penetration, the increase may be limited when the retinol-loaded, porous delivery vehicle is incorporated into an oil/water emulsion carrier, such as in the case of Comparative Example B. Unexpectedly, the combination of an anhydrous delivery system containing retinol-loaded, porous delivery vehicle and a carrier having a low water content, such as in the case of Example 1, results in a more significant overall increase in retinol penetration. Such significant overall increase associated with Example 1 is surprising and unexpected because typically, compositions that are anhydrous or have a low water content tend to be thick and/or have a relatively high viscosity, which can potentially trap the active, such as retinol, near the skin surface and/or hinder the delivery of the active into the deeper regions of the skin. Without intending to be bound by theory, the unexpected and surprising overall increase associated with Example 1 may be attributed to the anhydrous or low-water-content characteristics of the carrier and/or the delivery system, which may improve the stability of the active, e.g., retinol, as the retinol penetrates the skin over time.

Comparative Example C showed a marked decrease in retinol delivery, especially in the epidermis. Although the carrier used in Comparative Example C was anhydrous and further included a structuring agent (polysilicone-11), it also had a high viscosity of about 110,000 cP, as compared to the viscosity of about 30,000 cP of the carrier in Example 1. Without intending to be bound by theory, the retinol in Comparative Example C was likely physically trapped within the much thicker carrier.

### Experiment 3—Buildup of Retinol Over Time

While Experiments 1 and 2 examined the total level of retinol penetration after three days of daily dosing, Experiment 3 sought to examine both the total level of retinol penetration after three days of daily dosing and the level of retinol penetration after each day, to determine the rate at which the final level of retinol was reached.

Three sets of samples were prepared and tested. For each set of samples, Comparative Example A, Comparative Example B, Comparative Example D, and Example 1 (collectively referred to as “the formulas”) were applied to NativeSkin® human skin explants (diameter of 1.1 cm) from Genoskin, Inc.

For a first set of samples, Comparative Example A, Comparative Example B, Comparative Example D, and Example 1 were applied at 0 hours, and then the retinol contents in the epidermis and dermis were determined at 24 hours via HPLC quantitative analysis for each sample.

For a second set of samples, Comparative Example A, Comparative Example B, Comparative Example D, and Example 1 were applied at 0 and 24 hours, and then the retinol contents in the epidermis and dermis were determined at 48 hours via HPLC quantitative analysis for each sample.

For a third set of samples, Comparative Example A, Comparative Example B, Comparative Example D, and Example 1 were applied at 0, 24, and 48 hours, and then the retinol contents in the epidermis and dermis were determined at 72 hours via HPLC quantitative analysis for each sample.

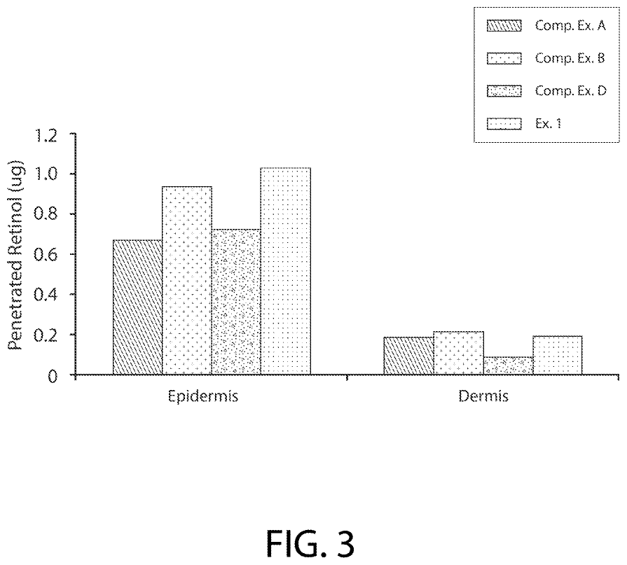

FIG. 3 shows retinol penetration, measured in μg of delivered retinol, in both the epidermis and dermis at 72 hours after three days of daily dosing. It is noted that in Experiment 1, medium sized skin explants (diameter of 1.5 cm) were used, while in Experiment 3, small sized skin explants (diameter of 1.1 cm) were used, likely resulting in the lower amount of retinol penetration observed for Comparative Example A in Experiment 3 than in Experiment 1.

Compared to Comparative Example A, Example 1 showed increased levels of retinol penetration, e.g., in the epidermis. In contrast, Comparative Example D exhibited a decreased level of retinol penetration in the skin, particularly in the deeper dermis region. Without intending to be bound by theory, the retinol in Comparative Example D was likely physically trapped within the thick carrier due to the high viscosity of 85,000 cP associated with the carrier used in Comparative Example D, hindering overall retinol penetration.

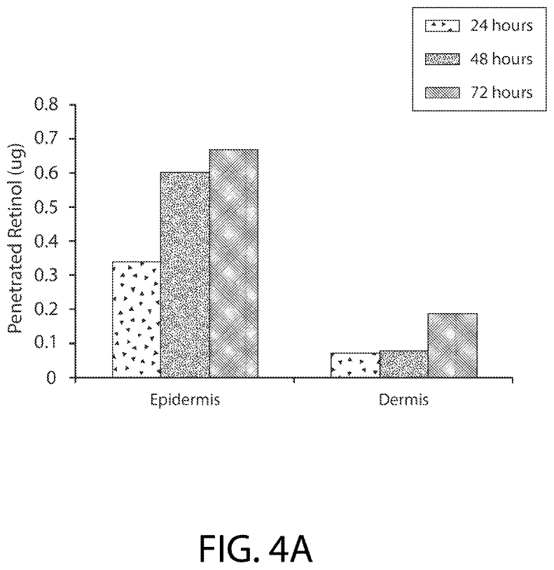

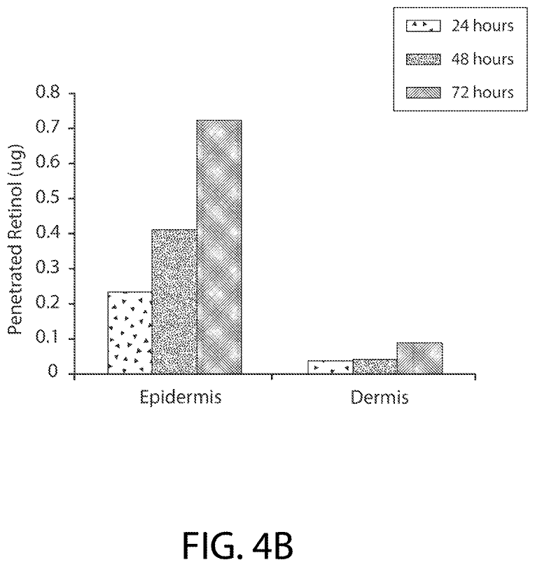

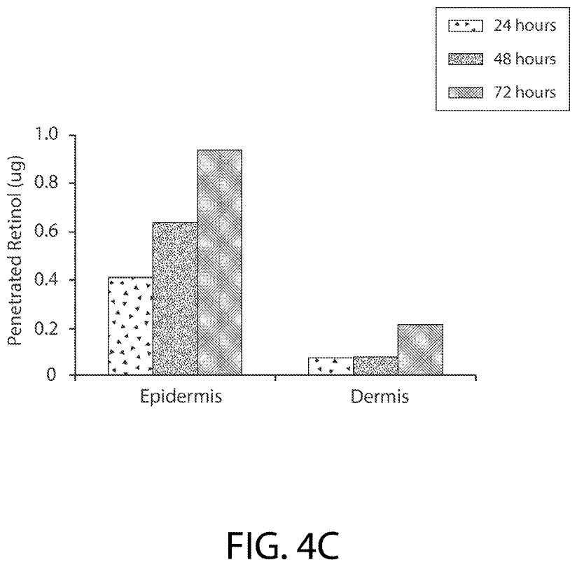

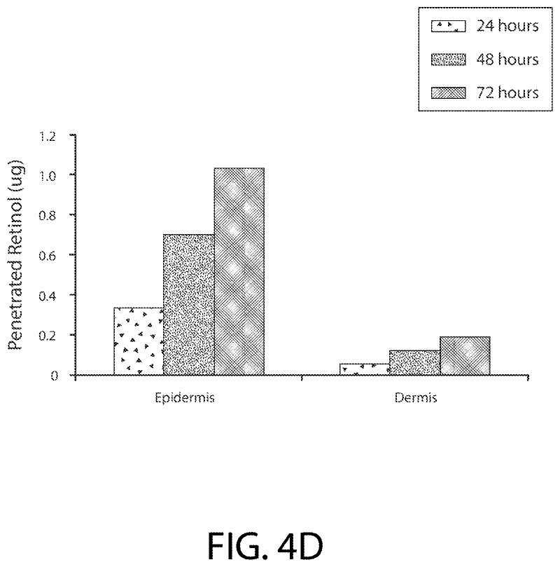

FIGS. 4A-4D show the buildup of retinol in both the epidermis and dermis associated with Comparative Example A, Comparative Example B, Comparative Example D, and Example 1, respectively. For each sample, the buildup of retinol is indicated by retinol in the epidermis and dermis at 24 hours after one dosing, at 48 hours after two daily dosing, and at 72 hours after three daily dosing. The inventors have unexpectedly discovered that the combination of an anhydrous delivery system in a carrier having a low water content, such as in the case of Example 1, achieves a surprisingly sustained (as opposed to burst) increase of retinol in both the epidermis and dermis over time.

FIG. 4A shows the buildup of retinol in both the epidermis and dermis associated with Comparative Example A. As shown, the increase in retinol in the epidermis appeared to plateau or level off by 72 hours. This suggests that a near maximum level of retinol was achieved in the epidermis after 48 hours and is more broadly suggestive of a burst release of retinol into the epidermis. In contrast, there is a more constant buildup of retinol in the epidermis for Comparative Example B, Comparative Example D, and Example 1, as shown in FIGS. 4B-4D, respectively, which suggests a more sustained release of retinol into the skin's epidermis.

Comparative Example B, Comparative Example D, and Example 1 all used a delivery system having retinol loaded in a porous delivery vehicle, specifically, Microsponge®, which form crosslinked polymer matrices, while Comparative Example A used a delivery system having retinol loaded in vesicles that self-assemble in water and held together by noncovalent forces. Thus, using a delivery system having a retinol-loaded, porous delivery vehicle, such as Microsponge®, can achieve a more sustained release of retinol into the skin's epidermis when compared to a delivery system having retinol loaded in self-assembled vesicles in water.

With respect to the buildup of retinol in the deeper dermis region of the skin, Comparative Example A, Comparative Example B, and Comparative Example D, as shown in FIGS. 4A-4C, respectively, all follow the same trend: there was virtually no change between 24 and 48 hours, followed by a marked increase in retinol at the final 72 hours. In contrast, with respect to Example 1, FIG. 4D shows a more sustained buildup of retinol in the dermis throughout the entire period tested.

As discussed above, it is desirable to achieve both a high overall level of retinol penetration and a sustained buildup over time as reaching the same level of retinol via a burst release can lead to visible skin irritation. Although Comparative Example B and Comparative Example D may achieve a sustained delivery of retinol into the epidermis as shown in FIGS. 4B and 4C, Example 1 where a retinol-loaded, porous delivery vehicle was incorporated in a carrier having a low water content is capable of achieving a sustained buildup of retinol in both the epidermis and dermis as shown in FIG. 4D.

In view of the results of Experiments 1 to 3, the inventor has unexpectedly discovered that the combination of an anhydrous delivery system having a retinol-loaded, porous delivery vehicle and a carrier having a low water content, such as in the case of Example 1, provides superior overall retinol delivery when compared to the combination of a delivery system having retinol-loaded in vesicles that self-assemble in water and an oil/water emulsion carrier. More importantly, incorporating a retinol-loaded, porous delivery vehicle, such as Microsponge®, in a carrier having a low water content provides the surprising results of sustained release of retinol into both the epidermis and the dermis, while delivering a higher overall level of retinol into the skin. In contrast, although the combination of a retinol-loaded, porous delivery vehicle and other carriers tested (such as the oil/water emulsion carrier in Comparative Example B or the carrier in Comparative Example D having a high viscosity) achieves a sustained delivery of retinol into the epidermis of the skin, these combinations exhibit a “burst-like” release of retinol into the dermis of the skin.

Additionally, the inventor has discovered that optimal performance (i.e., increased and sustained retinol delivery) can be achieved when the retinol-loaded, porous delivery vehicle is incorporated in a carrier that has both a low water content (i.e., less than 35 wt. % of water based on the weight of the carrier or even being anhydrous) and a suitable viscosity ranging from about 6,200 cP to about 60,000 cP, such as a viscosity of about 30,000 cP in the case of Example 1. Carriers that have much higher viscosities may hinder retinol delivery, such as the carrier of Comparative Example C and the carrier of Comparative Example D having a viscosity of about 110,000 cP and about 85,000 cP, respectively.

A second non-limiting exemplary personal care composition (Example 2) and a fifth comparative personal care composition (Comparative Example E) were prepared as follows.

### Example 2

A second non-limiting exemplary personal care composition (Example 2) was prepared in accordance with aspects of the invention. The second exemplary personal care composition included a delivery system having retinol loaded in a porous delivery vehicle, specifically, Microsponge®, and a carrier with a low water content. The loading amount of the retinol was 20 wt. % to 25 wt. % based on a total weight of the delivery system, and the delivery system included substantially no water. The carrier included no more than 35 wt. % of water based on a total weight of the carrier. The carrier further included a dimethicone/vinyl dimethicone crosspolymer as a structuring agent and had a viscosity of about 6,200 mPa·s (or 6,200 cP). The carrier included substantially no decamethylcyclopentasiloxane (D5) (or less than 0.1 wt. % of D5). Further, the carrier included substantially no parabens (or less than 0.1 wt. % of parabens). The amounts of the retinol-containing delivery system and the carrier were selected such that the resultant second exemplary personal care composition included about 0.5 wt. % of retinol and no more than 35 wt. % of water based on a total weight of the second exemplary personal care composition.

### Comparative Example E

A fifth comparative personal care composition (Comparative Example E) was prepared as follows. The fifth comparative personal care composition included a delivery system having retinol loaded in vesicles formed through self-assembly of amphiphilic surfactants in water. The fifth comparative personal care composition further included an oil/water emulsion as the carrier incorporating the delivery system. Thus, both the delivery system and the carrier included water in the fifth comparative personal care composition. The carrier had a viscosity of about 16,550 to about 18,500 cP. The carrier further included decamethylcyclopentasiloxane (D5). The fifth comparative personal care composition included about 0.5 wt. % of retinol and 57.46 wt. % of water based on a total weight of the fifth comparative personal care composition.

### Experiment 4

In vitro penetration tests (IVPT) of retinol delivery were conducted on NativeSkin® human skin explants from Genoskin Inc. Specifically, at 0, 24, and 48 hours, 12 μL of the personal care composition of Example 2 was applied to the center of a skin piece and rubbed on for 10 seconds, and 12 μL of the personal care composition of Comparative Example E was applied to the center of another skin piece and rubbed on for 10 seconds. N=4 samples were used for both of Example 2 and Comparative Example E. At 72 hours, retinol contents in the skin explants were measured in the epidermis and dermis using HPLC quantitative analysis.

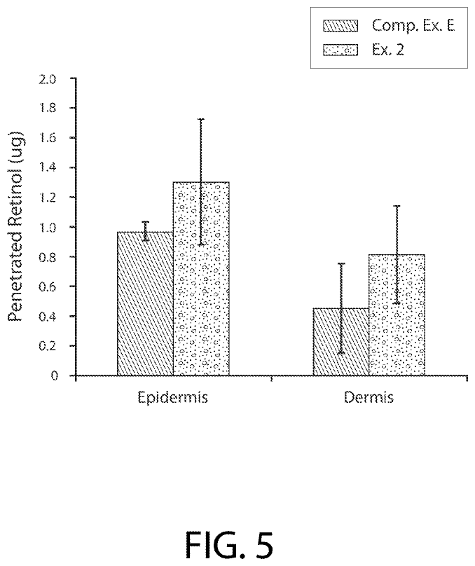

FIG. 5 shows the total retinol contents in regions of the skin for Example 2 and Comparative Example E. The numerical values are shown in Table 3 below.

TABLE 3
Retinol Contents in Regions of Skin

| | Epidermis | Dermis |
|---|---|---|
| Comparative Example E | 0.97 ± 0.06 μg | 0.45 ± 0.30 μg |
| Example 2 | 1.30 ± 0.42 μg | 0.81 ± 0.33 μg |

The inventor has unexpectedly discovered that the carrier of Example 2, which has a low water content and is free of D5 and parabens, can successfully delivers retinol past the outermost portion of the skin and into more biologically relevant regions. More significantly, Example 2 delivered about 30% more retinol into the epidermis than Comparative Example E. In the deeper dermis layer of the skin, Example 2 had about 80% more retinol delivered when compared to Comparative Example E.

In view of the above, a carrier that has a low water content (or even being anhydrous) and is free of D5 and/or parabens can be utilized to incorporate a delivery system having a retinol-loaded, porous delivery vehicle, such as Microsponge®, for effective retinol delivery. This combination allows for improved overall retinol delivery compared with the combination of retinol-loaded vesicles that self-assemble in water and a carrier that incorporates large amounts of both water and D5.

1. A personal care composition, comprising:
a delivery system comprising a retinoid, or derivative thereof, incorporated in a delivery vehicle; and
a cosmetically acceptable carrier comprising no more than about 35 wt. % of water;
wherein the personal care composition comprises no more than about 35 wt. % of water, based on a total weight of the personal care composition.

2. (canceled)

3. The personal care composition according to claim 1, wherein the cosmetically acceptable carrier is substantially anhydrous, preferably anhydrous.

4. The personal care composition according to claim 1, wherein the cosmetically acceptable carrier comprises a structuring agent, wherein the structuring agent comprises a polymer, a wax, or a combination thereof.

5. The personal care composition according to claim 4, wherein the structuring agent comprises a wax selected from silicone wax, paraffin wax, microcrystalline wax other than paraffin, polyethylene wax, and a combination of two or more thereof.

6. The personal care composition according to claim 4, wherein the structuring agent comprises a polymer selected from silicone elastomers.

7. The personal care composition according to claim 6, wherein the silicone elastomer is selected from dimethicone crosspolymer, dimethicone/vinyl dimethicone crosspolymer, polysilicone-11, and a combination of two or more thereof, optionally wherein the silicone elastomer has a viscosity of about 6,200 cP to about 50,000 cP.

8. The personal care composition according to claim 4, wherein the structuring agent is present in an amount ranging from about 5 wt. % to about 65 wt. % based on the total weight of the personal care composition.

9. The personal care composition according to claim 1, wherein the cosmetically acceptable carrier comprises a silicone oil, optionally wherein the silicone oil is present in an amount ranging from about 55 wt. % to about 95 wt. % based on the total weight of the personal care composition.

10. The personal care composition according to claim 9, wherein the silicone oil is selected from dimethicone, cyclopentasiloxane, cyclomethicone, phenyl trimethicone, trimethylsilylamodimethicone, stearoxytrimethylsilane, a polydimethylsiloxanes, a polydimethylsiloxane, a phenyl silicone, and a combination of two or more thereof.

11. The personal care composition according to claim 1, wherein the personal care composition is substantially free of decamethylcyclopentasiloxane, preferably free of decamethylcyclopentasiloxane.

12. The personal care composition according to claim 1, wherein the cosmetically acceptable carrier comprises a nonionic surfactant, optionally wherein the nonionic surfactant comprises at least one of laureth-4, laureth-23, C12-14 alketh-12, polysorbates surfactants, polyethoxy fatty ethers surfactants, or sorbitan fatty acid esters surfactants.

13. The personal care composition according to claim 1, wherein the retinoid, or derivative thereof, comprises retinol, optionally wherein the retinoid, or derivative thereof, is present in an amount ranging from about 0.05 wt. % to about 1 wt. % based on the total weight of the personal care composition.

14. The personal care composition according to claim 1, wherein the delivery vehicle comprises a plurality of porous structures, optionally wherein the plurality of porous spheres have an average size ranging from about 10 μm to about 20 μm.

15. The personal care composition according to claim 14, wherein the plurality of porous structures comprises an acrylate crosspolymer.

16. The personal care composition according to claim 14, wherein the plurality of porous structures comprises at least one of a methyl methacrylate/glycol dimethacrylate crosspolymer, cellulose, allyl methacrylates crosspolymer, or lauryl methacrylate/glycol dimethacrylate crosspolymer.

17. The personal care composition according to claim 1, wherein the delivery system further comprises at least one of a vitamin A, a vitamin C, a penetration enhancer, an antioxidant (e.g., propyl gallate, butylated hydroxytoluene (BHT), or a combination thereof), a surfactant, or a combination thereof, incorporated in the delivery vehicle.

18. The personal care composition according to claim 1, wherein the personal care composition is a controlled release personal care composition, optionally at a sustained rate of delivery.

19. A personal care composition, comprising:
a cosmetically acceptable carrier; and
a delivery system comprising:
a polymeric matrix; and
a retinoid, or a derivative thereof;
wherein the personal care composition comprises no more than about 20 wt. % water, based on the total weight of the personal care composition; and
wherein the polymeric matrix comprises a release-rate controlling polymer in an amount effective to control release of the retinoid.

20. A method of treating a skin condition comprising applying a personal care composition according to claim 1, to a skin surface of a subject in need thereof.
